(19) United States
(12) Patent Application Publication
(10) Pub. No.: US 2026/0115715 A1
(43) Pub. Date: Apr. 30, 2026

(54) DIAGNOSTIC CARTRIDGE

(71) Applicant: Hemex Health, Inc., Portland, OR (US)

(72) Inventors: Peter Galen, Portland, OR (US); James Daren Bledsoe, Portland, OR (US); Stephanie Bledsoe, Portland, OR (US); Steven M. Goss, Salem, OR (US); Matthew Lind, Portland, OR (US); David John Sayler, Portland, OR (US); Jered Wikander, Portland, OR (US)

(21) Appl. No.: 18/681,458

(22) PCT Filed: Aug. 8, 2022
(86) PCT No.: PCT/US2022/074671

§ 371 Date: Feb. 5, 2024

Related U.S. Application Data
(60) Provisional application No. 63/230,593, filed on Aug. 6, 2021.

(51) Int. Cl. B01L 3/00; A61B 5/15; B01L 9/00

(52) U.S. Cl. CPC B01L 3/502715; A61B 5/150022; A61B 5/150213; A61B 5/150251; A61B 5/150755; B01L 3/50273; B01L 3/523; B01L 9/52; B01L 2200/025; B01L 2200/0605; B01L 2200/0621; B01L 2200/10; B01L 2200/16; B01L 2300/044; B01L 2300/0672; B01L 2300/0822

(57) ABSTRACT

The disclosed diagnostic test cartridges collect a controlled amount of a patient sample using a capillary slide. The capillary slide mates with a capillary channel to cause diluent to mix in a controlled manner with the patient sample to prepare it for further processing, such as sonication and diagnostic testing. The cartridge can be a two-part design in some examples that selectively couple together to cause the patient sample to be collected in a controlled manner and the diluent to be released to mix with the patient sample in a controlled manner to improve the quality of the prepared sample for diagnostic testing.

### CROSS-REFERENCE TO RELATED APPLICATIONS

This application is a 371 filing of international application PCT/US22/74671, filed Aug. 8, 2022, which claims priority and benefit from the U.S. Provisional Patent Application 63/230,593, filed Aug. 6, 2021, and titled, “DIAGNOSTIC CARTRIDGE,” which are incorporated herein by reference in their entirety for all purposes.

### BACKGROUND

Patient diagnostic services save lives, reduce the time to treatment for the patient and provide valuable insight for targeted treatment. In many developed countries, modern medical facilities can provide patients with the most advanced diagnostic services allowing patients to be efficiently and effectively treated. In less developed countries or regions, high quality medical facilities and diagnostic services can be lacking, often due to economic and infrastructure considerations. In many less developed countries, the economy cannot afford the latest in medical technology and infrastructure, such as a robust power grid or highly trained clinicians, required to support the high demands of modern medical technology. Sadly, a large portion of the world's population resides in underserved or developed areas where the lack of efficient and effective diagnostic services critically impacts the population morbidity, mortality and overall health. This lack of medical care can lead or contribute to knock-on effects, such as low economic and educational development.

Less developed countries and areas lack significant diagnostic services that could shorten treatment and increase the living standard of the population. Many of the diseases and conditions the populations of less developed countries and areas face have largely been extinguished in developed countries, which means the treatment exists, may be plentiful, and may be, in some cases, relatively low cost for these diseases and conditions. The component that is lacking is the diagnostic services to diagnose members of the population effectively and efficiently so that they can receive prompt, timely treatment, which minimizes the impact of the disease or condition on the patient and the population.

Often, many less developed countries and areas also lack sufficient trained users that are typically required to perform the necessary diagnostic services. This can lead to inconclusive or erroneous results from diagnostic services or to significant delays in diagnosis as the diagnostic services are required to be performed in another location that has the requisite infrastructure and/or knowledge to perform the diagnostic service. For patients, this can mean further delays in treatment, which can decrease their chances of survival, increase the spread of the disease, and/or lead to increased debilitation caused by the disease or condition.

Often, many less developed countries and areas also lack the educational development that is typically required to perform the necessary diagnostic services. This can lead to inconclusive or erroneous results from diagnostic services or to significant delays in diagnosis as the diagnostic services are required to be performed in another location that has the requisite infrastructure and/or knowledge to perform the diagnostic service. For patients, this can mean further delays in treatment, which can decrease their chances of survival, increase the spread of the disease, and/or lead to increased debilitation caused by the disease or condition.

One of the common diseases effecting less developed countries and areas is malaria, a disease caused by a mosquito-borne parasite, plasmodium. Malaria infects many people each year, disproportionately in less developed countries and areas than developed ones. Malaria, if identified at an early stage of the infection, can be easily treated with relatively low cost treatment plans, but without early diagnosis, the disease causes great harm to individual patients, it quickly spreads among a population, and later-stage treatment is often costly and less effective. The populations most effected by malaria are vulnerable and do not have good access to quality and timely diagnostic services. Further, the malaria disease is very treatable if timely detected or diagnosed, however, the diagnostic services needed are often not readily and/or easily available in the countries and areas in which malaria is endemic.

Many countries effected by malaria and many humanitarian aid groups have directed resources and technology to malaria control and reducing and managing the disease and others like it. These resources and technology attack malaria on two fronts, the control of mosquitoes and the treatment and diagnosis of the disease. The current gold standard diagnostic services used to diagnose malaria, such as polymerase chain reaction (PCR) based tests, are expensive and require sophisticated laboratory analysis, and point-of-care blood films (light microscopy) and antigen-based rapid diagnostic tests (RDTs) lack the necessary sensitivity and speed to provide the necessary information to optimally treat malaria.

Where large laboratories may be prohibitively expensive and difficult to staff, point-of-care diagnostic devices may provide an effective solution. Such a solution could provide timely, accurate, and cost-effective health care.

### BRIEF DESCRIPTION OF THE DRAWINGS

FIG. 1 shows a block diagram of a sample diagnostic cartridge in accordance with the disclosure.

FIG. 2A shows side plan view of an example diagnostic cartridge in accordance with the disclosure.

FIG. 2B shows an exploded view of the example diagnostic cartridge shown in FIG. 2A.

FIG. 3 shows a method of using an example diagnostic cartridge according to the disclosure.

FIGS. 4A-B show a plan view, perspective view, and side view of the cartridge shown in FIGS. 2A-2B.

FIGS. 5A-5D show a perspective, side plan, cross-section of the side plan, and bottom plan view of the capillary slide of the example cartridge shown in FIGS. 2A-2B.

FIG. 6 shows an exploded view of the example cartridge shown in FIGS. 2A-2B.

FIG. 7 shows the example cartridge of FIGS. 2A-2B enclosed in an example vapor barrier.

### DETAILED DESCRIPTION

Cartridges for diagnostic devices and methods of using the cartridges are disclosed. The disclosed cartridges pick up a controlled amount of fluid, such as a patient biological sample like blood. The pickup of the controlled amount of patient sample, for example, to provide optimal levels of mixing of the patient sample with diluent. Such optimal levels of mixing then provide gold standard levels of prepared samples for sonication of the sample to lyse the cells and improve overall accuracy and precision of the diagnostic results.

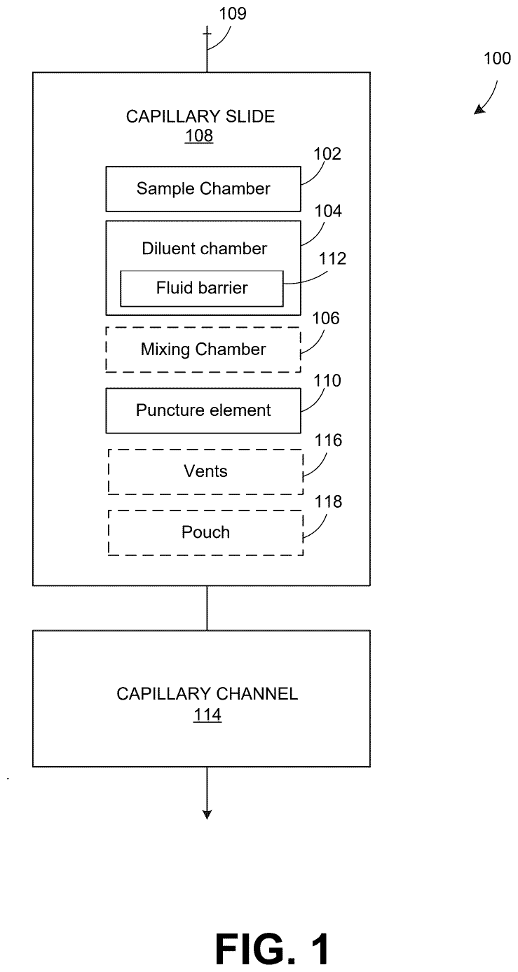

The cartridge 100 shown in FIG. 1 receives or obtains a fluid sample into a sample chamber 102. The cartridge 100 also has a diluent chamber 104 that stores diluent. Diluent is released to mix with the sample to produce a prepared sample that flows into or is mixed within a chamber, such as a mixing chamber 106 or the sample chamber 102. The cartridge 100 has a capillary slide 108 that is moveable within and with respect to a longitudinal axis of a mating capillary channel 114. The capillary slide 108 in some examples, includes a moveable piston that moves along the longitudinal axis. When the piston moves along the longitudinal axis, a back pressure is developed in the capillary slide 108. As the diluent flushes out of the capillary slide 108, a lumen and a sample drop off vent control the amount of diluent fluid that flows from the diluent chamber along with controlling the back pressure produced when the diluent flows from the capillary slide 108. The shape and contour of the lumen impact the manner in which the diluent flows from the capillary 108 as the lumen is a tunnel or tubular structure through which the diluent flows from the capillary slide 108. The sample drop off vent is an opening in the capillary slide and in some examples a dispensing end of the lumen as it releases diluent from the capillary slide 108, which can also control the flow rate of the diluent from the capillary slide 108. Further, the sample pick vents located at a pick-up end of the lumen opposite the dispensing end close during motion of delivering the diluent (the piston moving along the longitudinal axis) so as to create back pressure in the diluent chamber as the piston moves into the diluent chamber.

Once mixed, the mixed patient sample is typically sonicated to lyse the cells or compounds within it to create a prepared patient sample. A diagnostic test can be performed on the prepared sample. In an example, the prepared sample can be exposed to a magnetic field while having a light source transmit light through it to obtain a light transmittance value of the prepared sample (not shown). The light transmittance value can indicate presence, absence, or a value of a component in the prepared sample. In this example, the target component is paramagnetic and sensitive to the applied magnetic field so that the components align when the magnetic field is applied. The light transmittance value differs for these samples when the magnetic field is applied compared to when it is not applied and/or from an empirically normal or baseline light transmittance value of the sample. The detected light transmittance value indicates presence, absence, volume, gradations, relative difference or levels of an infection, parasite, disease condition, or other patient sample abnormality. Detecting the target component of the prepared sample can be done in any suitable way.

The cartridge 100 is a mating two-part design that, when the two parts are mated, causes diluent to be released and mixed with the sample. The two-part design can be two discrete parts that couple together or selectively attach together, such as by a snap fit or other mechanical connector. The action of coupling the two parts of the cartridge 100 creates the action of releasing diluent to mix with a patient sample to create a prepared patient sample for diagnostic testing.

In one example, the first part of the cartridge 100 includes a capillary slide 108 with a puncture element 110 and a fluid diluent chamber with a fluid barrier 112. The capillary slide 108 is positioned to move along a longitudinal axis 109 within a capillary channel 114. The capillary slide 108 and then capillary channel 114 can be any suitable shape that allows for the capillary slide 108 to move along the longitudinal axis 109 within the capillary channel 114. In an example, the capillary slide 108 and the capillary channel 114 have a mating annular shape although they can also have a mating cylindrical shape. The mating annular shape can be tapered to cause the capillary slide 108 to have a fixed distance it can move along the longitudinal axis 109 of the capillary channel 114 when the tapered end of the capillary slide 108 meets the tapered end of the capillary channel 114. In the two-part design, the capillary slide 108 and capillary channel 114 are in the first part while the mixing chamber 106 or the patient sample chamber 102-wherever the prepared patient sample is stored for diagnostic testing-is in the second part of the cartridge 100.

Optionally, the capillary slide 108 can include one or more vents 116 that help control the volume of patient sample that is collected for diagnostic purposes. These vents 116 prevent back pressure in the capillary slide 108 by allowing air to be released through the vents 116 when the patient sample is being collected and pulled into the capillary slide 108. The vents 116 are closed or covered when the capillary slide 108 is moved to the second position in which the piercing element 110 pierces the fluid barrier 112 of the diluent chamber 104. In the second position in which the vents 116 are closed or covered, the fluid of the patient sample or the diluent is unable to flow out of the vent, which prevents dispensing of any fluid in an undesired direction.

Also optionally, the diluent in the diluent chamber 104 or the diluent chamber 104 itself can include a pouch 118 that helps preserve the diluent until the piercing element 110 pierces or punctures the fluid barrier 112 of the diluent chamber 104 to release the diluent. The pouch 118 can also be punctured or pierced by the piercing element 110 when the cartridge capillary slide 108 is moved from the first position to the second position. Alternatively, the pouch 118 can enclose the capillary slide 108 entirely or portions of it to preserve the diluent. In this alternate example, the pouch 118 can be manually removed by a user before using the cartridge 100. The pouch 118 increases shelf life because it controls the evaporation of the diluent while the cartridge 100 is stored. The pouch 118 also increases the cleanliness of storing the cartridge 100 because it helps to collect any leaking diluent if the diluent chamber 104 or the fluid barrier 112 were to fail. Alternatively, the diluent can be stored in an ampule in a precise amount that is pierced by the piercing element to release the diluent for mixing with the patient sample.

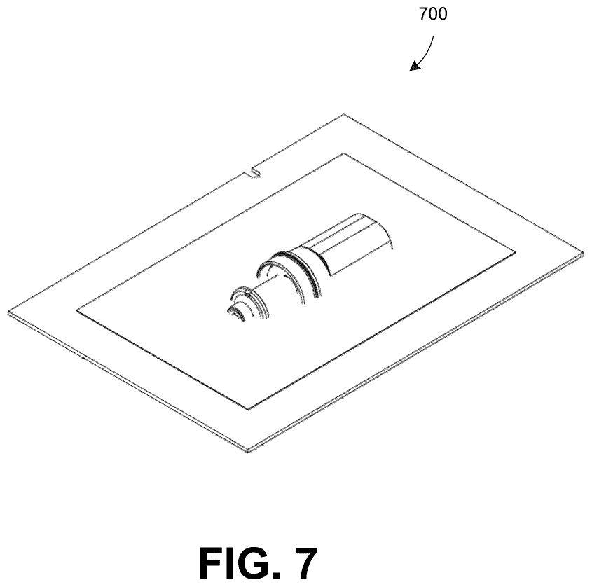

In some examples, the entire cartridge 100 can be enclosed in a vapor barrier 700, such as a bag or pouch, such as the example cartridge shown enclosed in a bag 700 in FIG. 7. The vapor barrier reduces the evaporation rate of fluid in the cartridge 100, which increases shelf life of the cartridge 100. Further, when the diluent is protected with such a vapor barrier the quality of the diluent remains at a high quality for a longer period of time, which prolongs degradation of the diluent and improves overall quality of the diluent over time when it comes time to release it to mix with the patient sample.

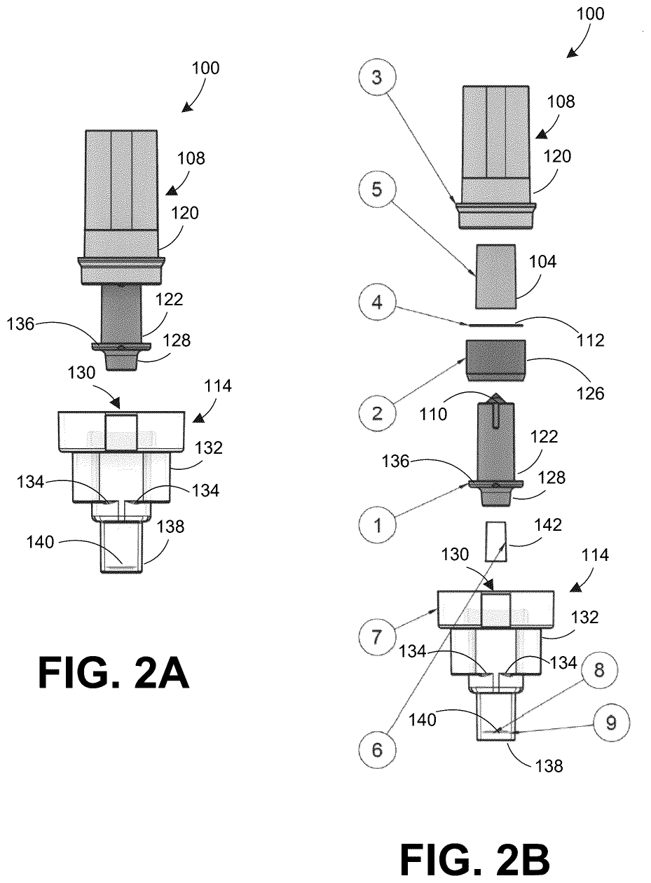

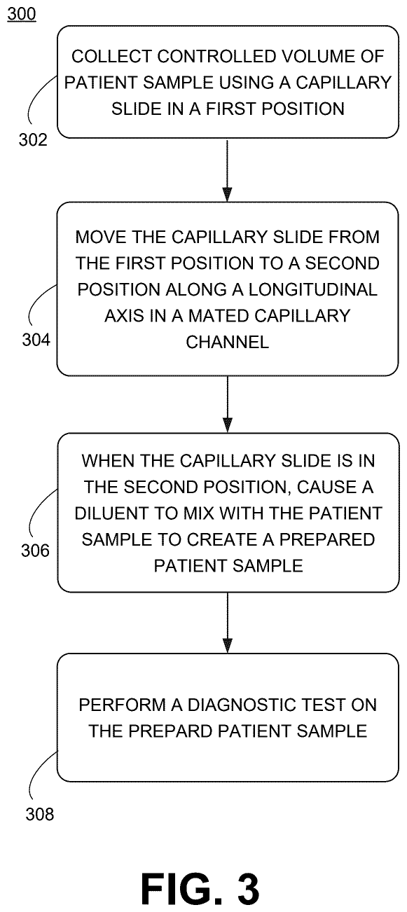

FIGS. 2A and 2B show an example cartridge 100 with the two-part design. FIG. 2A shows the capillary slide 108 and the capillary channel 114 in a first position in which the capillary slide 108 receives the patient sample and before the fluid barrier 112 is punctured by the piercing element 110. In this example, the capillary slide 108 has a handle 120 and a piston 122. The piston 122 of the capillary slide 108 collects or picks up the controlled volume of the patient sample in this example although in other examples the capillary slide 108 collects or picks up the patient sample. Here, the capillary slide 108 is shown in an annular embodiment with the piston 122 is also being annular so that they both mate with the annular capillary channel 114. In the two-part cartridge 100 design, when the capillary slide 108 is moved from the first position to the second position, the piston 122 with its piercing element 110 pierces the fluid barrier 112 and moves into the diluent chamber 104. Additional vents can be included on the piston 122 on the mating surface between the cuvette and the capillary slide that allows air to escape from the cuvette to avoid back pressure in the cuvette. These vents are located on the outer side wall of the piston 122 and create a channel on this outside piston wall that run partial to full length of the piston 122 to allow air to escape the diluent chamber as the piston 122 moves along the longitudinal axis. In some examples, the released diluent and the patient sample are flushed into a cuvette when this occurs. The sonication of the sample and diagnostic testing can be performed on sample in the cuvette.

FIG. 2B shows an exploded view of the cartridge 100 embodiment shown in FIG. 2A. The diluent chamber 104 is an ampule that stores diluent and is positioned within the handle 120 of the capillary slide 108. The piercing element 110 of the capillary slide 108 is positioned on an end of the piston 122 that is nearest the diluent chamber 104. The cartridge 100 shown in FIG. 2B has a fluid barrier 112 that is an ampule membrane positioned between the piercing element and the diluent chamber. The diluent chamber 104 and the fluid barrier 112 are surrounded by a gasket 126 that helps maintain the positioning of the diluent chamber 104 and the fluid barrier 112 and fluid seal within the handle 120 of the capillary slide 108. The gasket 126 also fits around the piercing element 110 of the piston 122.

The handle 120 can include the diluent chamber 104 and can be sized to match the volume of the prepared sample, the diluent, or the patient sample. The volume size of the diluent chamber 104 in the handle 120 can help manage motion force to reduce as much air in the diluent chamber and/or air mixing with diluent as it is released to mix with the patient sample. This optimal removal of air mixing with the diluent prevents air from acting as a spring with creates irregularities in the diluent or as the diluent mixes with the patient sample.

In an example, the capillary slide 108 can be removed from the capillary channel 114 entirely to allow the prepared patient sample to be subjected to diagnostic testing that would otherwise be impeded by the capillary slide 108, such as the capillary slide 108 being a physical space constraint to the particular type of diagnostic test. The capillary slide 108 could be separated from the capillary channel 114 for any suitable reason or at any desired time after the patient sample is prepared in the patient chamber or more specifically in the cuvette that may be located in the capillary channel 114.

In the example with the diluent chamber 104 in the handle 120, diluent can be added to the diluent chamber 104 in excess of the volume of diluent that is intended to be released to mix with the patient sample. The excess diluent is present to help offset any evaporation of diluent that occurred during storage, which helps extend shelf life of the cartridge. Further, the diluent chamber 104—whether in the handle 120 or elsewhere in the cartridge 100—and any portion of the cartridge through which the diluent flows have smooth, contoured surfaces to avoid any edges or sharp features over which diluent flows. This smooth, contoured flow path for the diluent prevents any existing air bubbles from being “cut” or separated into smaller air bubbles that are attracted to and easier to mix with the diluent, which is undesirable to maintain a high quality diluent and mixing process.

The piston 122 has a tapered end 128 that has a smaller diameter than the rest of the piston 122. The tapered end 128 of the piston 122 fits into a cavity 130 within the capillary channel 114. The cavity 130 tapers as well or decreases its diameter within a tapered portion 132 to mate with the tapered end 128 of the piston 122 when the capillary slide 108 is moved to the second position. The cavity 130 of the capillary channel 114 also has two shoulders 134 that provide a mechanical stop when the piston 122 is moved to the second position. A collar 136 of the piston 122 abuts the shoulders 134 of the cavity 120 of the capillary channel 114 when the capillary slide 108 is moved to the second position.

The capillary piston 122 of the capillary slide 108 is annular and is slidably moveable within the mating annular shape of the capillary channel 114 from a first position to a second position in which its piercing element 110 pierces the fluid barrier of the diluent chamber 104. Piercing the fluid barrier releases the diluent stored within the diluent chamber 104 to mix with the patient sample. In some example, the piercing of the fluid barrier releases the diluent into a mixing chamber 106 to flush the patient sample, which causes the diluent and the patient sample to mix. In other examples, the piercing of the fluid barrier releases the diluent into the capillary slide 108 for the same flushing process. In yet other examples, the capillary slide 108 has a patient sample chamber 102 into which the fluid diluent is released. In still other examples, the capillary slide 108 has a mixing chamber 106 that is separate from both the patient sample chamber 102 and the diluent chamber 104 and into which the diluent is released and the patient sample is released. In this last example, the patient sample can be released prior to, simultaneously with, or after the diluent is released. In the example shown in FIGS. 2A and 2B, the capillary channel 114 has a cuvette 138 that receives the mixed diluent and patient sample. The diluent and patient sample are mixed in the cuvette 138 to create the “prepared patient sample.” Sonication beads 140, such as glass beads, suspended in a bead solution can be included in the cuvette to held sonicate the prepared sample in the cuvette 138, such as the example embodiment shown in FIGS. 2A and 2B. In the examples with sonication beads, the diluent and patient sample are deposited into the cuvette 138 that stores the sonication beads 140 suspended in the bead solution.

The capillary slide can have one or more vents 116 that are open when the capillary slide 108 is receiving the patient sample. The vents 116 allow pressure change, reducing or avoiding back pressure that would otherwise be caused when the sample is received into the capillary slide 108. These same vents 116 close when the capillary slide 108 is moved to a second position in which the piercing element 110 pierces the fluid barrier 112. The closed vents 116 prevent premature release of the patient sample-diluent mixture or prepared sample. The vents 116 can be can one or more openings within a shaft of the piston 122 of the capillary slide 108, such as on the outside wall progressing towards the capillary channel 108.

As explained above, the capillary slide 108 can have a variable length when it receives or collects the patient sample. The piston 122 of the capillary slide 108 can have multiple positions within the handle 120 that adjusts the exposed length of the piston 122, for example. Some examples can alternatively have capillary slides of a fixed length. Those fixed length capillary slides can be offered in variable lengths. Varying the length of the capillary slide 108 controls the volume of the patient sample that is received. For example, a first length of the capillary slide 108 exposes a first surface area of the capillary slide 108 to the patient sample while a second length of the capillary slide 108 exposes a second surface area of the capillary slide that differs from the first surface area. Additionally, the first length could expose certain surface features of the surface area of the capillary slide 108 to the patient sample that differs from the second length, which could expose more or fewer surface features that help or prevent receipt of the patient sample.

For example, the piston 122 length can be varied by physically changing the dimensions of the piston 122 to a different size for different applications. A different sized piston determines a different volume of patient sample that is collected or diluent that is delivered to mix with the patient sample, which varies based on the diagnostic test, for example, or for other reasons. The different sized pistons 122 can be interchanged by a user for different applications, as needed. The size of the individual pistons is fixed but can be interchanged at the user's discretion.

As discussed above, the piercing element 110 pierces the fluid barrier 112, which can be a polypropylene membrane for example, as shown in FIG. 2B. The piercing element 110 pierces the fluid barrier 112 along a tear path when the capillary slide 108 is moved from the first position to the second position. The tear path is a direction, contour, shape, or the like of the manner in which the fluid barrier 112 separates to create an opening through which diluent flows. The piercing element 110 can be a selected shape or size to control the manner in which the fluid barrier 112 is pierced, which in turn influences the tear path. For example, an edge of the piercing element 110 can be tapered to a point or a thin blade. A pointed piercing element 110 pierces the fluid barrier 112 in a shape of its point, which creates a tear path from that point while a thin bladed piercing element 110 pierces the fluid barrier along a linear slice of its blade shape, which creates a different tear path from that linear slice.

In another example, the pointed piercing element 110 can be a spire in a spiral shape that wraps around the piston 122. The spiral shape helps to control the tear path in a continue spiral as the piston 122 pierces through the fluid barrier 112. The spire on the piston 122 can act

Alternatively, a spire could be included in the interior cavity of the handle 120 of the capillary slide 108 such that it still interacts with the piston 122 as the piston moves from the first position to the second position. The combination of the spire and the piston 122 act as a capillary to promote diluent flow through the capillary slide in a similar manner to the embodiment with the spire located on the piston 122.

The piercing element 110 can also be asymmetrical or can be annular or semi-annular in other examples. Any variation in shape, size, contour, or other features of the piercing element 112 influences the tear path accordingly because it directly varies the force, shape, and pierce point of the piercing element 110 as it first contacts the fluid barrier 112. That first pierce point helps define the tear path of the fluid barrier 112 as the fluid barrier 112 is pierced and subsequently torn. The fluid barrier 112 can also have a predefined tear path that helps guide the manner in which it separates to create the opening such as perforations that helps ease the separate of the fluid barrier 112 in a desired direction after the piercing element first pierces the fluid barrier 112. The perforations in the fluid barrier 112 and work in tandem with the shape, contour, size, etc. of the piercing element 110 to precisely control the tear path.

The diluent chamber can be located anywhere in the capillary element, such as in the handle. The diluent chamber can also have two parts-one that stores the fluid diluent and a second that receive gaseous diluent that evaporates from the fluid diluent. In some examples, the gaseous diluent is recycled back into the chamber that stores the fluid diluent under certain conditions like adding heat or to the ambient environment of the cartridge before its use.

Alternatively, the cartridge includes a fluid regulating element that helps to regulate fluid flow of the released diluent into the sample or as it mixes with the sample. The fluid regulating element can be placed in either of the two parts to control fluid flow of the diluent, the sample, the mixing of the diluent and sample, the prepared sample (a mixture of the diluent and sample), or a combination of these. As discussed above, the first part has a sample chamber that stores the fluid sample and a diluent chamber that stores the diluent. A fluid barrier separates the fluid sample chamber and the diluent chamber. When the first part is mated with the second part, the fluid regulating element moves to a position in which it pierces or is forced to break through the fluid barrier, which separates the sample chamber from the diluent chamber of the first part to result in mixing of the sample fluid and diluent.

The capillary slide 108 can also have a coating 142 on it. One example is a lubricating coating that helps to reduce the friction between the capillary slide 108 and the capillary channel 114 when the capillary slide 108 moves within it. Another example is an anti-coagulating coating that includes an anti-coagulation agent that helps prevent the patient sample or the prepared patient sample from coagulating.

The capillary channel 114 can include one or more mechanical elements that contact a portion of the capillary slide 108 when it is moved within the capillary channel 114, such as a shoulder like those shown in FIG. 2B or another mechanical stop such as a mating tapered shape. The mechanical elements can be mechanical stops that prevent the capillary slide 108 from move beyond a particular location in the capillary channel 114. Such mechanical stops position the capillary slide 108 in a position to release the prepared sample into a mixing chamber 106, such as the cuvette shown in FIGS. 2A and 2B for example. In this example, once the prepared patient sample is in the cuvette, it can undergo light transmittance analysis. The cuvette is only one example chamber in which the prepared sample can undergo light transmittance analysis and other configurations can be used.

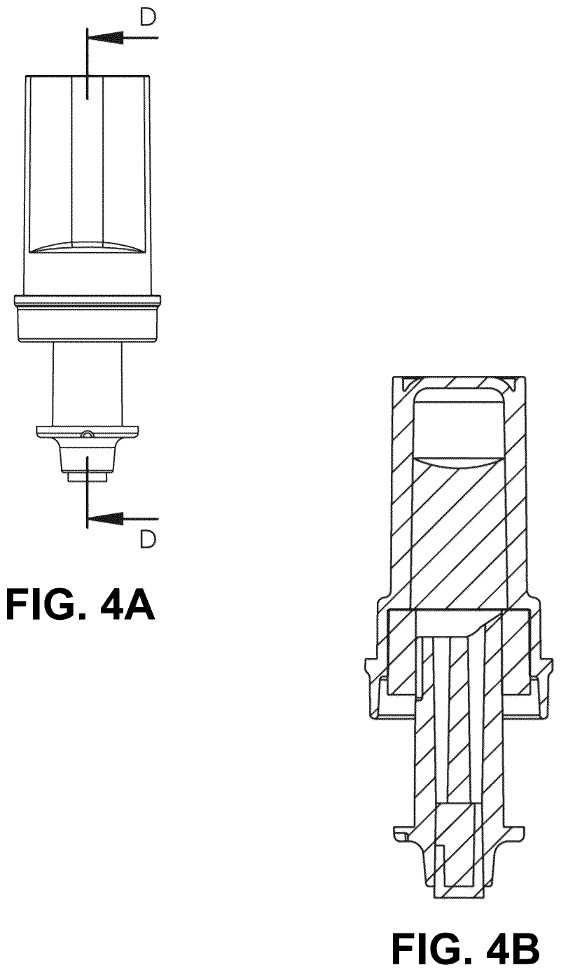

Turning now to FIG. 3, the disclosed cartridges can be used to collect patient samples for diagnostic testing. While the capillary slide is in the first position, it collects a controlled volume of the patient sample 302. The capillary slide is moved from the first position to a second position along a longitudinal axis in a mated capillary channel 304. In some of the examples discussed herein, the capillary slide is mated within the capillary channel. When the capillary slide is in the second position, a diluent is caused to mix with the collected patient sample to create a prepared patient sample 306. For example, moving the capillary slide to the second position causes diluent to be released from a diluent chamber and mixed with the collected patient sample. A diagnostic test can then be performed on the prepared patient sample 308. In the examples discussed herein, the prepared patient sample could be mixed and prepared for lysing within a cuvette that is then exposed to a magnetic field to detect magnetic or paramagnetic compounds, such as iron, in the patient sample. As discussed above, light can be transmitted through the prepared sample with and without the applied magnetic field to determine a light transmission values for each state of the prepared sample. Diagnostic results can be made from analyzing the light transmission values and/or their relative values with respect to each other or empirical data.

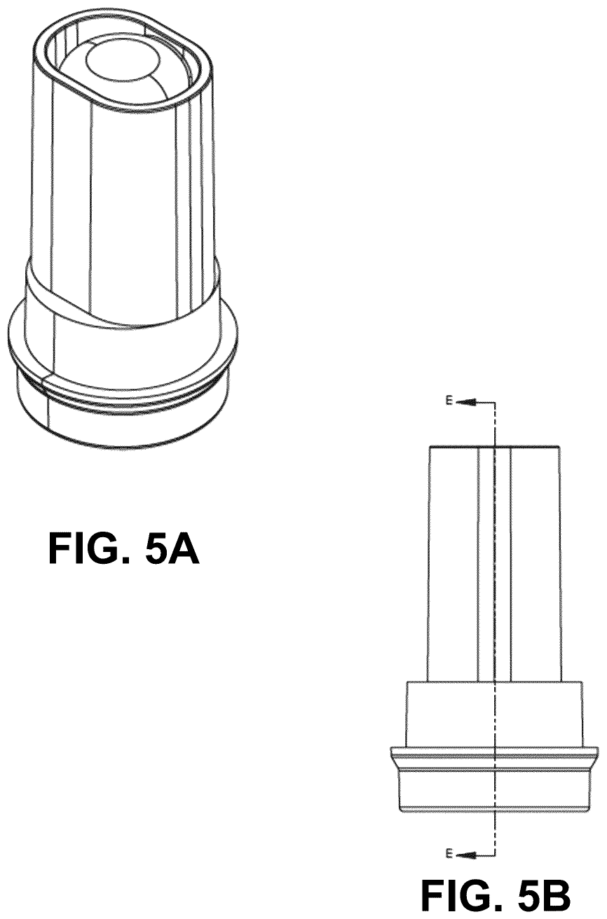

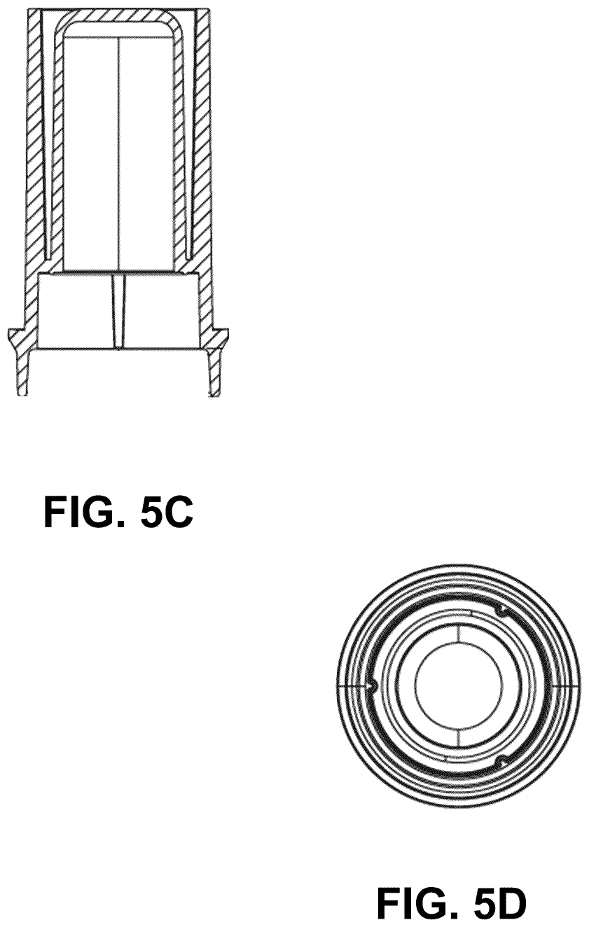

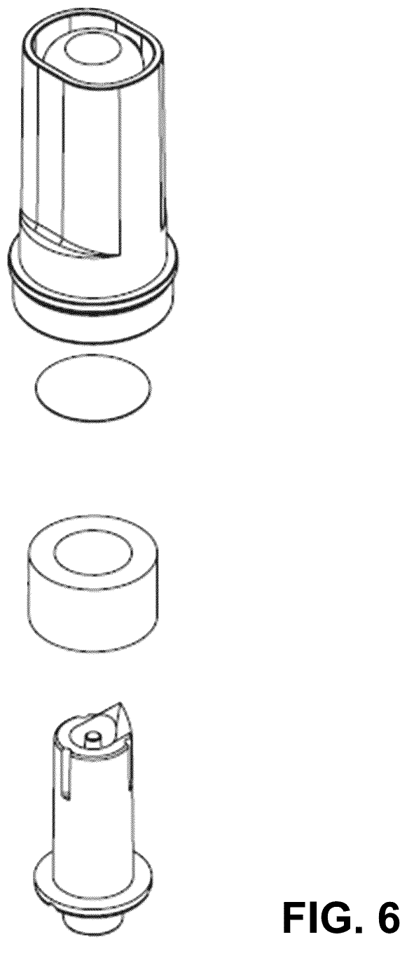

FIGS. 4A-4B show a side plan and cross-section of the side plan taken along D-D, respectively of the cartridge shown above in FIGS. 2A-2B. FIG. 5A-D showing a perspective, side view, cross-section of the side view, and a bottom plan view of the capillary slide shown in FIGS. 2A-2B. FIG. 6 shows an exploded perspective view of the cartridge shown in FIGS. 2A-2B. FIG. 7 shows the cartridge shown in FIGS. 2A and 2B.

In another example, the first part of the cartridge has two portions that move with respect to each other from a first, extended position to a second, retracted position. When this movement occurs, the fluid regulator pierces or is forced to break the fluid barrier and the fluid mixing occurs. The movement of the two portions can include moving one or both of the two portions along an axis. As the movement occurs, a portion of one portion that has the fluid regulator extends through the fluid barrier and into a hollow second portion that includes the mixing chamber and/or the sample chamber in the examples shown in the attached figures.

In an example, the fluid regulating element is a spire formed along an annular periphery of an interior of the first part of the cartridge. The fluid regulating element can be positioned within the diluent chamber of the first part, in some examples. Alternatively, the fluid regulating element can be positioned outside of the diluent chamber, between the sample chamber and the diluent chamber, or within the sample chamber. When the first and second parts are mated, the spire pierces the fluid barrier or forces it to break, which causes the diluent to flow in a controlled fashion guided by the piercing location of the fluid barrier. The targeted piercing of the fluid barrier causes the diluent to flow into a mixing chamber or the sample chamber through the point of piercing the fluid barrier and is governed by the size and shape of the pierce point and the manner in which the fluid barrier tears after it is pierced. The fluid flow dynamics can be manipulated by the location and size of the pierce point and the tear pathway of the fluid barrier. Without the controlled flow, bubbles can result from the mixing of the diluent and the fluid samples, which decreases the quality of the prepared sample that is later tested.

The flow of the diluent into the mixing chamber in the example shown in the attached figures causes it to flow over a ring geometry of the spire's surface, which controls both the direction and rate of the fluid flow as the sample fluid and diluent mix together. This controlled flow of diluent to mix with the sample fluid allows these fluids to mix during the mating of the two cartridge parts before bubbles rise into the mixing chamber. The spire can further provide a mechanical stop to prevent fluid flow by plugging the flow pathway if it is shaped to be fitted within the interior walls of the mixing chamber, such as with a compression fit or plug structure, for example. Alternatively, a shelf or other mechanical stopping mechanism provides a physical limit to how far the spire with the fluid regulator can extend into the mixing chamber or sample chamber.

Further, the disclosed cartridges can include one or more features that help break, tear, puncture, or otherwise remove a portion or all of the fluid barrier between the fluid sample chamber and the diluent chamber. For example, the cartridge includes a tip that pierces the fluid barrier in a specific location to cause the fluid barrier to preferentially tear along an expected pathway or axis. The tip could be a post that is either sharpened or rounded at the end that pierces the fluid barrier. In an example, the tip is positioned along an annular periphery of a sample acquisition portion, such as the capillary shown in the attached figures. The portion of the first part of the cartridge with the diluent chamber is moved along an axis to fit over the sample chamber portion of the first part. The physical movement of the two portions with respect to each other causes the fluid barrier to be moved towards the end of the capillary with the piercing tip (or alternatively the opposite direction of movement of the two portions or a combination of moving the portions together). The piercing tip contacts the fluid barrier when this movement occurs and pierces the fluid barrier to release the diluent fluid, which then mixes with the sample fluid. The location and shape of the piercing tip controls the location at which the fluid barrier is pierced. In the illustrated example, this piercing point is at a location along an annular periphery of the fluid barrier.

After the fluid barrier is pierced, it tears along a tear pathway that affects the fluid dynamics of the released diluent. The tear can also be controlled by a lancing profile that further guides the tear along a particular tear pathway. For example, the desired lancing profile is annular and extends along the periphery of the fluid barrier. The lancing profile could be an edge that extends around some portion or all of the periphery of the end of the capillary that includes the piercing tip. The lancing profile can have a sharpened, rounded, square, or other shaped edge. The lancing profile could be the same height through its entire length or could taper in height through its length. For example, the attached figures show a lancing profile that is a sharpened edge that is positioned next to the piercing tip and tapers and it extends around a portion of the annular periphery of the capillary. This configuration causes the piercing tip to pierce the fluid barrier and the lancing profile to encourage and guide the tear of the fluid barrier along its annular periphery. The piercing location and annular periphery tear pathway produce a ring geometry of the fluid flow.

The first and second portion of the first part of the disclosed cartridge can also include a guide or locking mechanism that secures the movement of the first part with the second part as one or both of them are moved into a position that pierces the fluid barrier. The guide can be a channel or cavity on one portion that has a mating protrusion on the other portion that causes the two portions to move along a single axis to control movement along other axes, such as a lateral direction. For example, the guide illustrated in the examples shown in the attached figures have a mating channel and protrusion arrangement. When the first portion and/or second portion are moved with respect to each other the protrusion fits within the channel to control lateral movement within a specific tolerance. The channel also has a stop that contacts an end of the protrusion at a desired distance. That desired distance is the controlled distance to move the two portions with respect to each other, which helps to control the piercing and tearing of the fluid barrier.

Although the subject matter has been described in language specific to structural features and/or methodological acts, it is to be understood that the subject matter defined in the appended claims is not necessarily limited to the specific features or acts described. Rather, the specific features and acts are disclosed as exemplary forms of implementing the claims.

We claim:

1. A diagnostic cartridge, comprising:
a capillary channel;
a capillary slide moveable from a first position in which the capillary slide is structured to collect a controlled volume of a patient sample to a second position along a longitudinal axis,
wherein a portion of the capillary slide is mated within the capillary channel when the capillary slide moves from the first position to the second position.

2. The cartridge of claim 1, wherein the capillary slide and the capillary channel have a mating annular shape.

3. The cartridge of claim 1, wherein the capillary slide and the capillary channel have a mating cylindrical shape.

4. The cartridge of claim 1, wherein the capillary slide and the capillary channel have a mating tapered end.

5. The cartridge of claim 1, further comprising a piercing element attached to the capillary slide.

6. The cartridge of claim 5, further comprising a diluent chamber having a fluid barrier, the piercing element positioned to puncture the fluid barrier when the capillary slide is moved to the second position.

7. The cartridge of claim 6, wherein, when the fluid barrier is punctured by the piercing element, the diluent stored in the diluent chamber is released into a mixing chamber, and the patient sample is also released into or present within the mixing chamber.

8. The cartridge of claim 6, wherein, when the fluid barrier is punctured by the piercing element, the diluent stored in the diluent chamber is released into a patient sample chamber in which the patient sample is stored after collection.

9. The cartridge of claim 6, wherein when the fluid barrier is punctured by the piercing element, the diluent stored in the diluent chamber is released to mix with the patient sample, the patient sample released before, after, or simultaneously with the diluent.

10. The cartridge of claim 6, wherein the piercing element punctures the fluid barrier along a tear path.

11. The cartridge of claim 6, wherein the diluent chamber has a fluid sub-chamber and a gas sub-chamber that are fluidly connected to each other to allow condensed gas to flow from the gas sub-chamber to the fluid sub-chamber.

12. The cartridge of claim 10, wherein the tear path is controlled by a shape or feature of the piercing element.

13. The cartridge of claim 5, wherein the piercing element includes a point or thin blade.

14. The cartridge of claim 5, wherein the piercing element is asymmetrical, annular, or semi-annular.

15. The cartridge of claim 1, wherein the capillary slide has a vent.

16. The cartridge of claim 14, wherein the vent is open when the capillary slide is in the first position and is closed when the capillary slide is in the second position.

17. The cartridge of claim 14, wherein the capillary slide has multiple vents, the multiple vents open when the capillary slide is in the first position and is closed when the capillary slide is in the second position.

18. The cartridge of claim 1, wherein a length of the capillary slide is variable.

19. The cartridge of claim 1, wherein the capillary slide is adjustable from a first length to a second length, the first length associated with a first exposed surface area of the capillary slide and the second length associated with a second exposed surface area that differs from the first exposed surface area.

20. The cartridge of claim 1, wherein the capillary slide has a lubricating or anti-coagulating coating.

21. The cartridge of claim 1, further comprising a capillary slide stop that prevents movement of the capillary slide along the longitudinal axis beyond the second position.

22. The cartridge of claim 1, further comprising a vapor barrier that encloses the cartridge.
